(19) United States
(12) Patent Application Publication
(10) Pub. No.: US 2016/0296190 A1
(43) Pub. Date: Oct. 13, 2016

(54) RADIOGRAPHIC IMAGE SHOOTING SYSTEM

(71) Applicant: Konica Minolta, Inc., Tokyo (JP)

(72) Inventors: Kenichirou SUZUKI, Tokyo (JP); Koji TAKEMURA, Tokyo (JP)

(21) Appl. No.: 15/091,979

(22) Filed: Apr. 6, 2016

(30) Foreign Application Priority Data
Apr. 7, 2015 (JP) 2015-078112

(51) Int. Cl. A61B 6/00

(52) U.S. Cl. CPC A61B 6/5241; A61B 6/461

(57) ABSTRACT

A radiographic image shooting system includes: a shooting stand configured to include a holder capable of being loaded with radiographic image shooting devices; an irradiation device configured to be capable of emitting radiation to the radiographic image shooting devices loaded in the holder; and a console configured to control shooting based on selected shooting order information and composite images acquired from the radiographic image shooting devices to produce a long image, wherein when the selected shooting order information is shooting order information, the console creates a first shooting condition key, the console determines the number of the images for use in the production of the long image, and creates newly a number of second shooting condition keys, and the console links the images acquired from the radiographic image shooting devices with the second shooting condition keys, and links the long image with the first shooting condition key.

The entire disclosure of Japanese Patent Application No. 2015-078112 filed on Apr. 7, 2015 including description, claims, drawings, and abstract are incorporated herein by reference in its entirety.

### BACKGROUND OF THE INVENTION

1. Field of the Invention

The present invention relates to a radiographic image shooting system, in particular, to a radiographic image shooting system including a shooting stand for one-shot long shooting.

2. Description of the Related Art

To radiographically shoot a relatively wide region of a patient's body such as the entire spine or the entire lower extremity by a radiographic image shooting device (Flat Panel Detector) F, conventionally as illustrated in FIG. 15A, for example, the radiographic image shooting device F loaded into a holder 101 is made movable along a support column 102 of a shooting stand 100, and an irradiation device 103 emits radioactive rays to a patient P as a subject to be shot while the radiographic image shooting device F is changed in position along the direction of a body axis A of the patient P (that is, vertical direction) to perform long shooting and obtain a plurality of radiographic images.

In the case of FIG. 15A, a collimator 104 having an opening not illustrated is disposed between the irradiation device 103 and the radiographic image shooting device F such that the collimator 104 is vertically moved in accordance with the movement of the radiographic image shooting device F to narrow a radiation field. Then, the thus shot radiographic images are put together to produce a long radiographic image (refer to JP 2013-154146 A, for example). Hereinafter, a long radiographic image will be referred to as long image.

In the case of FIG. 15A, however, while the irradiation device 103 emits a plurality of radioactive rays to shoot a plurality of radiographic images with the radiographic image shooting device F changed in position, the patient P as a subject may moves in many cases, thereby leading to a body motion problem. When body motion is seen in any one of the radiographic images, it is difficult to obtain an appropriate long image even by re-shooting only the radiographic image and combining all the radiographic images. Accordingly, it is necessary to shoot again all the radiographic images (that is, perform again long shooting) to increase the exposed dose of the patient.

Accordingly, at present, there is being developed a shooting stand 200 in which a plurality of radiographic image shooting devices F1 to F3 is aligned in advance in a holder 201 along the direction of the body axis A of the patient P as illustrated in FIG. 15B, for example (refer to JP 2012-045159 A and others, for example). According to the configuration of this shooting stand, the irradiation device 103 can shoot a plurality of radiographic images by emitting a radiative ray only once (that is, at one shot) to the radiographic image shooting devices F1 to F3, without having to move these devices along the direction of the body axis A of the patient P, thereby causing no body motion problem.

As illustrated in FIG. 15B, the shooting stand in which a plurality of radiographic image shooting devices F can be loaded into a holder such that long shooting can be performed by emitting a radioactive ray only once to the radiographic image shooting devices F loaded in the holder will be referred to as shooting stand for one-shot long shooting.

To perform long shooting at one shot as illustrated in FIG. 15B, shooting order information as illustrated in FIG. 16 is prepared before the shooting. The shooting order information is generated by specifying at least a patient's name P3, a shooting region P7, and others. The shooting order information and others will be described later. The shooting order information with the shooting region P7 specified as “entire spine” or “entire lower extremity” constitutes shooting order information indicative of long shooting.

Conventionally, the operator such as a radiographer selects shooting order information indicative of long shooting with the shooting region P7 specified as “front side of entire lower extremity” from all pieces of shooting order information illustrated in FIG. 16, for example, as the next shooting target, and then creates shooting condition keys for specifying the segmented parts of the “front side of entire lower extremity” at the shooting region P7 to perform long shooting corresponding to the selected shooting order information.

The shooting condition keys constitute shooting information including the patient's name P3, the shooting region P7, and others, as the shooting order information does. For example, to perform long shooting using three radiographic image shooting devices, the operator such as a radiographer creates in advance three shooting condition keys “upper front side (of entire lower extremity),” “middle front side,” and “lower front side” as segmented parts of the shooting region P7 in correspondence with the foregoing one shooting order information for long shooting (for example, the shooting order information with the shooting region P7 as “entre lower extremity”).

For example, when the patient is a child or an infant, two radiographic image shooting devices F may be aligned to shoot the patient's entire lower extremity. In this case, the radiographer creates two shooting condition keys “upper front side (of entire lower extremity)” and “middle front side” or “middle front side (of entire lower extremity)” and “lower front side” as segmented parts of the shooting region P7 in correspondence with the foregoing one shooting order information for long shooting.

As described above, the shooting order information is shooting information generated at each shooting (that is, one piece of information at one-time shooting), and the shooting condition key is shooting information generated for each image (that is, one key for one image).

It is very troublesome for the operator such as a radiographer to create manually the shooting condition keys. Accordingly, the operator may select the shooting order information indicative of long shooting with the “entire lower extremity” specified on a selection screen H1 displayed on a display unit Ca of a console C (see FIG. 17) as described above, for example, to display a setting screen H2 for easy setting of the shooting condition keys as illustrated in FIG. 18, for example.

In this case, to perform long shooting using three radiographic image shooting devices F, the operator such as a radiographer can easily set the shooting condition keys by clicking button icons h21 indicative of “upper front side,” “middle front side,” and “lower front side” of “entire lower extremity” on the setting screen H2. To perform long shooting using two radiographic image shooting devices F, the operator can easily set the shooting condition keys by clicking the button icons h21 indicative of “upper front side” and “middle front side” or “middle front side” and “lower front side” of “entire lower extremity” on the setting screen H2.

To set further easily the shooting condition keys, although not illustrated, instead of setting the upper, middle, and lower parts on the setting screen H2 as described above, the operator such as a radiographer may enter only the number of the radiographic image shooting devices F (for example two, three, or the like) to be loaded into the holder at the time of long shooting such as shooting of “entire lower extremity”, for example.

Nevertheless, even though the shooting condition keys can be easily set as described above, the operator such as a radiographer still needs to create or set in advance a number of shooting condition keys according to the number of the radiographic image shooting devices F.

In some cases, the operator initially estimated that long shooting could be performed by two radiographic image shooting devices F, but after all, they find that three are necessary according to the body size of the patient or the like. In contrast, the operator estimated that three radiographic image shooting devices F would be needed but two are actually enough. In other cases, the operator attempted to perform long shooting by three radiographic image shooting devices F, but only two of them can be used because the remaining one is under fault conditions or the like.

In the foregoing cases, the operator such as a radiographer needs to create or set again the shooting condition keys. In the conventional radiographic image shooting system capable of long shooting, the operator cannot perform long shooting without doing troublesome work such as creating or re-creating the shooting condition keys according to the number of the radiographic image shooting devices F for actual use in long shooting.

In this regard, in simple shooting with only one-time radiation to one radiographic image shooting device F, the image to be obtained through simple shooting specified by one shooting order information is one and the number of the shooting condition key to be created is one. Accordingly, in the case of simple shooting, in general, when the operator such as a radiographer selects the one shooting order information, the console automatically creates the one shooting condition key corresponding to the shooting order information.

Accordingly, in the case of simple shooting, the operator such as a radiographer can perform shooting without having to consider the creation of the shooting condition key before the shooting or even being conscious of the console's automatic creation of the shooting condition key. In addition, in the case of simple shooting, only one radiographic image shooting device F is used for the shooting, and thus the operator such as a radiographer can perform shooting without having to consider the number of the radiographic image shooting device F for use in the shooting.

Accordingly, in the case of performing one-shot long shooting as illustrated in FIG. 15B, if the radiographic image shooting system is configured such that the operator such as a radiographer can perform long shooting without having to consider the number of the radiographic image shooting device(s) F for use in the shooting or doing troublesome work such as creating the shooting condition keys before the shooting as in the case of simple shooting, the operator such as a radiographer can perform long shooting with the operational feeling as if performing simple shooting. Accordingly, the radiographic image shooting system becomes very easy to use for the operator.

### SUMMARY OF THE INVENTION

The present invention has been made in view of the foregoing problems, and an object thereof is to provide a radiographic image shooting system that allows the operator such as a radiographer to perform one-shot long shooting with the operational feeling as if performing simple shooting, without having to consider the number of the radiographic image shooting devices for use in the one-shot long shooting.

To achieve the abovementioned object, according to an aspect, a radiographic image shooting system reflecting one aspect of the present invention comprises: a shooting stand configured to include a holder capable of being loaded with a plurality of radiographic image shooting devices; an irradiation device configured to be capable of emitting radiation to the plurality of radiographic image shooting devices loaded in the holder; and a console configured to control shooting based on selected shooting order information and composite images acquired from the plurality of radiographic image shooting devices to produce a long image, wherein when the selected shooting order information is shooting order information for long shooting, the console creates a first shooting condition key for the long shooting, the console determines the number of the images for use in the production of the long image, and creates newly a number of second shooting condition keys corresponding to the determined number of the images in association with loading positions of the radiographic image shooting devices having shot the images in the holder, and the console links the images acquired from the radiographic image shooting devices with the second shooting condition keys corresponding to the loading positions of the radiographic image shooting devices, and links the long image produced by compositing the images with the first shooting condition key.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is a diagram showing a configuration of a radiographic image shooting system of an embodiment;

FIG. 2 is a diagram showing a configuration example of the radiographic image shooting system in which a plurality of shooting rooms and one or more consoles are associated with one another;

FIG. 3 is a diagram showing another configuration example of a one-shot long shooting stand;

FIG. 4A is a diagram showing an exposure switch in an irradiation device, FIG. 4B is a diagram showing the halfway-pressed button, and FIG. 4C is a diagram showing the fully-pressed button;

FIG. 5 is a perspective view showing the outer appearance of the radiographic image shooting device;

FIG. 6 is a block diagram showing an equivalent circuit in the radiographic image shooting device;

FIG. 7A is a diagram showing dongles arranged at individual loading positions in a holder of the shooting stand, and FIG. 7B is a diagram showing the state in which the dongle is connected to a connector of the radiographic image shooting device;

FIG. 8 is a diagram showing an example of a display screen displayed on a display unit of a console;

FIG. 9 is a timing chart for describing timings for the process of resetting a radiation detection element, electric charge accumulating state, application of an on voltage to each scanning line in the process of reading image data, and the like;

FIG. 10 is a timing chart for describing timings for application of the on voltage to each scanning line before start of the process of reading offset data;

FIG. 11 is a diagram showing the state in which preview images are displayed on the display screen;

FIG. 12 is a diagram describing that each of generated images is linked with second shooting condition keys;

FIG. 13A is a diagram describing composition of the images, and FIG. 13B is a diagram describing that a composite long image is associated with a first shooting condition key;

FIG. 14 is a diagram showing the state in which the composite long image is displayed on the display screen;

FIG. 15A is a diagram describing a conventional method for long shooting, and FIG. 15B is a diagram describing a method for one-shot long shooting;

FIG. 16 is a diagram showing an example of shooting order information;

FIG. 17 is a diagram showing an example of a selection screen for selecting shooting order information; and

FIG. 18 is a diagram showing an example of a setting screen for setting shooting condition keys.

### DESCRIPTION OF THE PREFERRED EMBODIMENTS

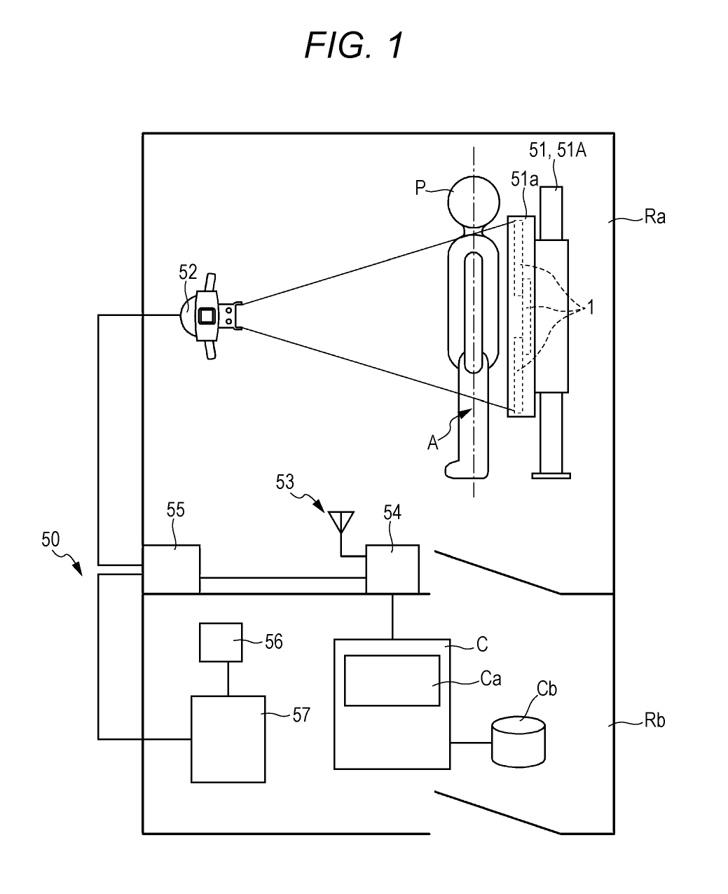

Hereinafter, an embodiment of a radiographic image shooting system according to the present invention will be described with reference to the drawings. However, the scope of the invention is not limited to the illustrated examples. FIG. 1 is a diagram showing a configuration of a radiographic image shooting system of the embodiment.

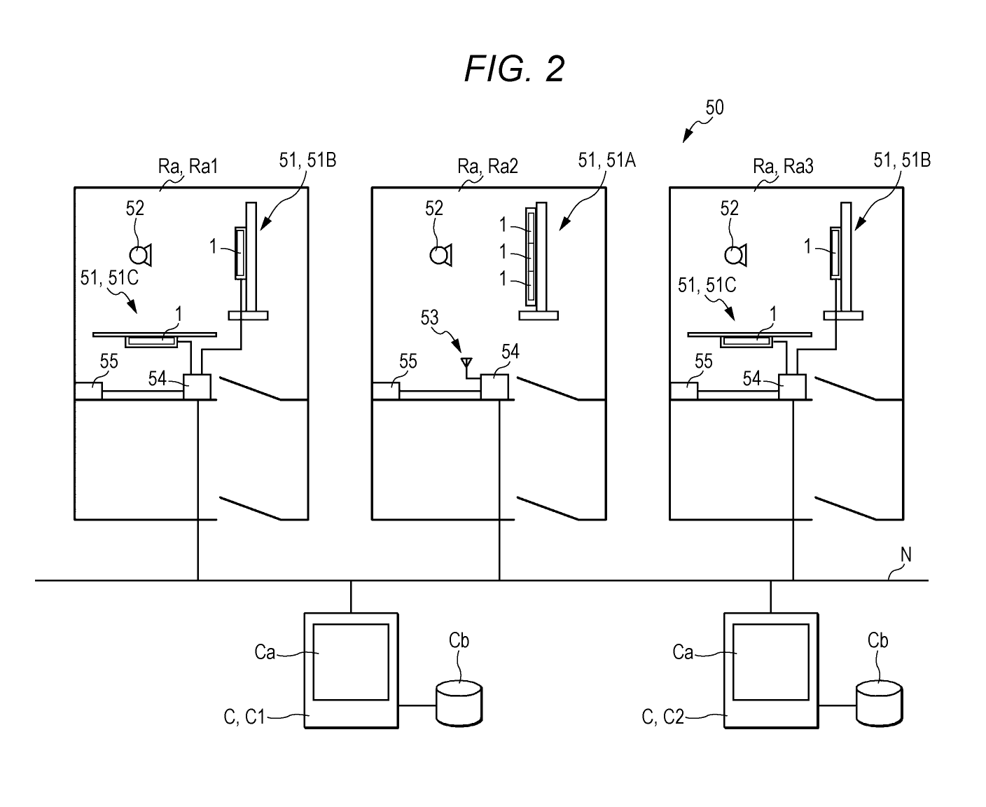

FIG. 1 shows only a shooting stand 51A for one-shot long shooting installed in a shooting room Ra. Besides, a shooting stand 51B for upright shooting to be used in simple shooting, a shooting stand 51C for decubitus shooting, and others may be installed in the shooting room Ra (see FIG. 2). That is, although the shooting stand 51A for one-shot long shooting needs to be installed in the one shooting room Ra, it can be decided as appropriate whether any other modalities are to be installed in the shooting room Ra.

The following descriptions of a basic configuration and others of a radiographic image shooting system 50 according to the embodiment are based on the assumption that the one shooting room Ra and one console C are associated with each other as illustrated in FIG. 1. Besides, a plurality of shooting rooms Ra (Ra1 to Ra3) and a plurality of consoles C (C1 and C2) may be associated with one another via a network N or the like as illustrated in FIG. 2.

When there are a plurality of shooting rooms Ra as illustrated in FIG. 2, the shooting stand 51A for one-shot long shooting needs to be installed in at least one of the shooting rooms Ra. Besides, it is possible to decide as appropriate what additional modalities are to be installed in the shooting room Ra and the others. The shooting stand 51A for one-shot long shooting may be installed in all of the shooting rooms Ra.

In the following descriptions, the shooting stand 51A for one-shot long shooting may also be referred to simply as shooting stand 51A. In addition, the following descriptions are based on the assumption that the shooting stand 51A for one-shot long shooting is used for upright shooting to shoot a patient P as a subject standing in front of the shooting stand 51A as illustrated in FIGS. 1 and 2. However, the shooting stand 51A for one-shot long shooting is not limited to this application. The present invention is also applicable to the case where, although not illustrated, the shooting stand 51A for one-shot long shooting is used for decubitus shooting to shoot the patient in a supine or seating position on the holder in which a plurality of radiographic image shooting devices is loaded.

[Basic Configuration of the Radiographic Image Shooting System]

As illustrated in FIG. 1, in the embodiment, the shooting stand 51A for one-shot long shooting capable of being loaded with a plurality of radiographic image shooting devices 1 is arranged in the shooting room Ra (when there is a plurality of shooting rooms Ra (see FIG. 2), at least one shooting room Ra is applied). The shooting stand 51A is configured such that the plurality of radiographic image shooting devices 1 aligned in the direction of the body axis A of the patient P as a subject can be loaded into a holder 51a.

The following descriptions are based on the assumption that the holder 51a of the shooting stand 51A can be loaded with three radiographic image shooting devices 1 as illustrated in FIGS. 1 and 2. In the present invention, however, the number of the radiographic image shooting devices 1 loaded into the shooting stand 51A is not limited to three but the number of loadable radiographic image shooting devices 1 may be two or four or more.

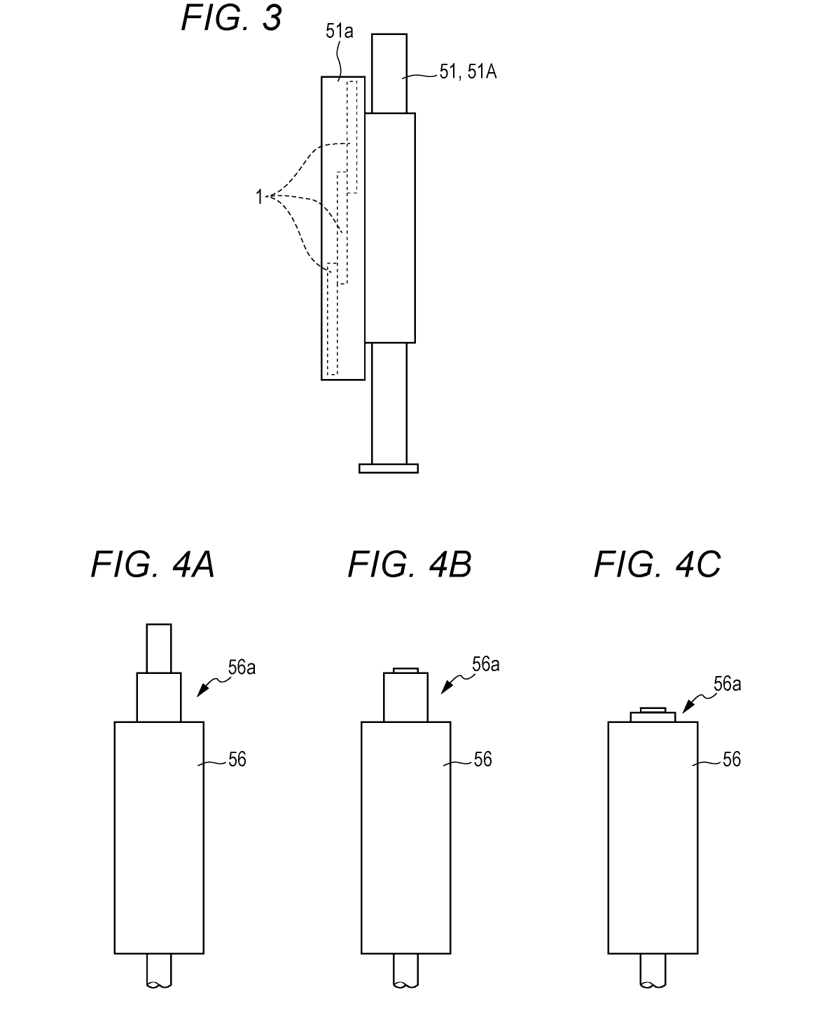

FIG. 1 shows the case where the radiographic image shooting devices 1 are loaded in the holder 51a alternately at the side close to an irradiation device 52 and the side distant from the irradiation device 52. For example, as illustrated in FIG. 3, the radiographic image shooting devices 1 may be loaded in the holder 51a such that the lower radiographic image shooting devices 1 are closer to the irradiation device 52 (see FIG. 1 and others) than the upper radiographic image shooting device 1.

The irradiation device 52 is installed in the shooting room Ra. As illustrated in FIG. 1, the irradiation device 52 for use in long shooting is a wide-angle irradiation type that emits a radiative ray once (that is, one shot) onto the radiographic image shooting devices 1 loaded in the shooting stand 52A through the patient P as a subject. The irradiation device 52 can also be used for simple shooting applications such as upright shooting and decubitus shooting. To perform simple shooting, the radiation field of the irradiation device 52 for long shooting may be limited by a collimator.

A relay 54 is installed in the shooting room Ra to relay communications between the devices and the like in the shooting room Ra and the devices and the like outside the shooting room Ra. The relay 54 is provided with an access point 53 so that the radiographic image shooting devices 1 can transmit or receive image data D, signals, and others in a wireless manner. FIGS. 1 and 2 show the state in which the radiographic image shooting devices 1 loaded in the holder 51a of the shooting stand 51A and the relay 54 communicate with each other in a wireless manner. Alternatively, the shooting stand 51A, the radiographic image shooting devices 1, and the relay 54 may be connected together via a cable or the like for wired communications.

The relay 54 is also connected to a control unit 55 of the irradiation device 52 and the console C. The relay 54 contains a converter not illustrated that converts signals for LAN (local area network) communications and others to be transmitted from the radiographic image shooting devices 1, the console C, or the like to the control unit 55 of the irradiation device 52 into signals for the control unit 55, or converts the signals reversely.

As illustrated in FIG. 1, an operator station 57 of the irradiation device 52 is installed in a front room (also called operation room or the like) Rb. The operator station 57 is provided with an exposure switch 56 for the operator such as a radiographer to instruct the irradiation device 52 to start irradiation or the like. The operator station 57 can be used to set tube voltage, tube current, irradiation time, and the like to the control unit 55 of the irradiation device 52. The tube voltage and others may also be set or changed on the console C.

As illustrated in FIG. 4A, the exposure switch 56 includes a button 56a. As illustrated in FIG. 4B, when the operator such as a radiographer performs a first operation (halfway-press) on the button 56a of the exposure switch 56, the control unit 55 starts the irradiation device 52. As illustrated in FIG. 4C, when the operator performs a second operation (full-press) on the button 56a of the exposure switch 56, the control unit 55 causes the irradiation device 52 to emit radiation. The radiation from the irradiation device 52 and others will be described later.

There is also installed in the front room Rb the console C composed of a computer or the like in which a CPU (central processing unit), a ROM (read only memory), a RAM (random access memory), an input/output interface, and others not illustrated are connected to a bus. When the radiographic image shooting system 50 is configured as illustrated in FIG. 2, the console C may be arranged outside the shooting room.

The console C is provided with a display unit Ca composed of a CRT (cathode ray tube) or an LCD (liquid crystal display), and includes an input unit such as a mouse or a keyboard not illustrated. The console C is connected to or contains a storage unit Cb composed of an HDD (Hard Disk Drive) or the like. Although not illustrated, the console C is connected to an HIS (hospital information system), a RIS (radiology information system), a PACS (picture archiving and communication system), or the like via the network N or the like.

[About the Radiographic Image Shooting Device]

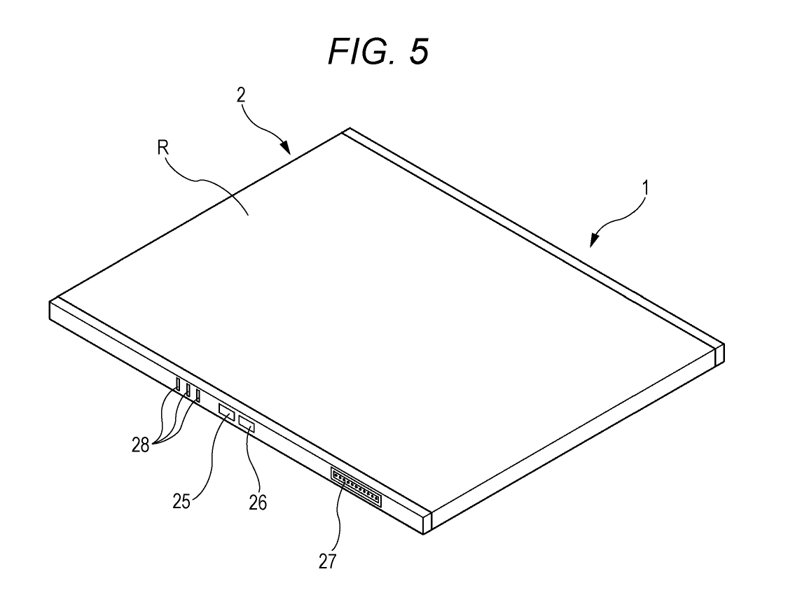

The radiographic image shooting device 1 for use in the radiographic image shooting system will be described. FIG. 5 is a perspective view of an outer appearance of the radiographic image shooting device.

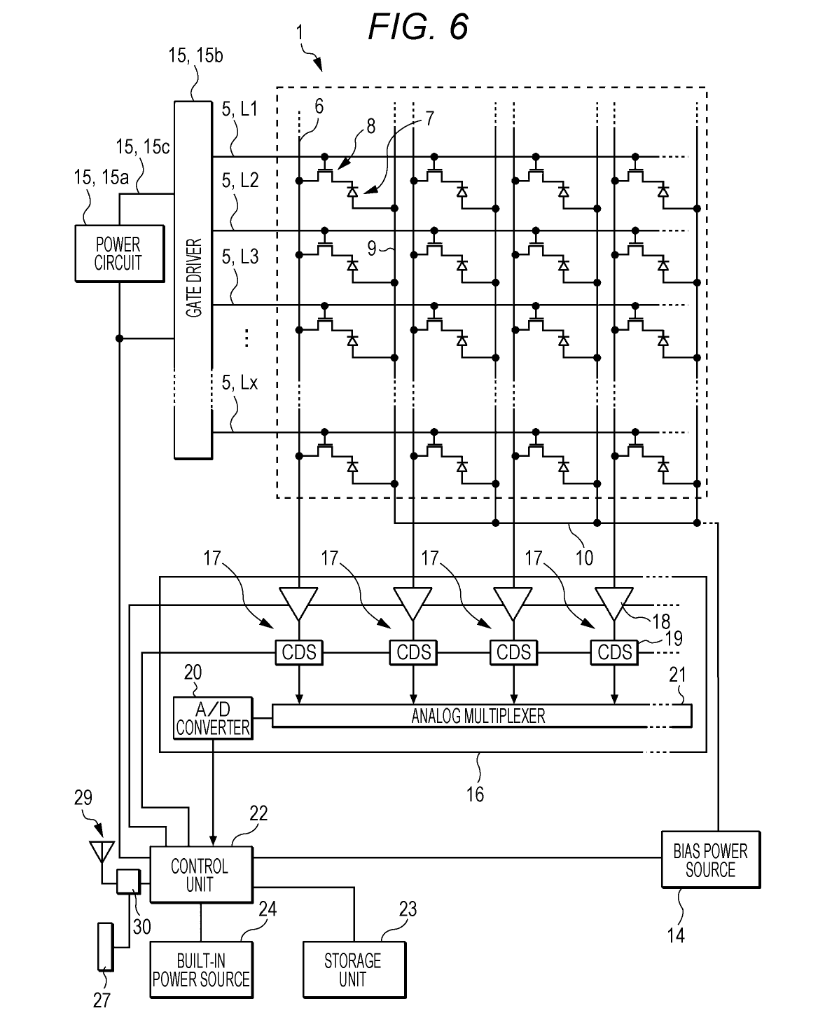

In the embodiment, the radiographic image shooting devices 1 is configured such that radiation detection elements 7 and others described later are stored in a casing 2, and a power switch 25, a changeover switch 26, a connector 27 as described above, an indicator 28, and others are arranged on one side surface of the casing 2. Although not illustrated, in the embodiment, an antenna 29 (see FIG. 6 described later) is provided on the opposite side surface or the like of the casing 2, for example, for wireless communications with the outside. For communications with the outside in a wired manner, a cable not illustrated can be connected to the connector 27.

FIG. 6 is a block diagram showing an equivalent circuit in the radiographic image shooting device. As illustrated in FIG. 6, the radiographic image shooting devices 1 has a plurality of radiation detection elements 7 aligned in a two-dimensional form (matrix) on a sensor substrate not illustrated. The radiation detection elements 7 generate electric charge according to the amount of emitted radiation. The radiation detection elements 7 are connected to a bias line 9, and the bias line 9 is connected to a connection line 10. The connection line 10 is connected to a bias power source 14 such that the bias power source 14 applies an inverse bias voltage to the radiation detection elements 7 via the bias line 9.

Each of the radiation detection elements 7 is connected to a thin film transistor (hereinafter, referred to as TFT) 8 as a switch element. The TFT 8 is connected to a signal line 6. A scanning drive unit 15 switches by a gate driver 15b between on voltage and off voltage supplied from a power circuit 15a via a wire 15c and applies the voltage to lines L1 to Lx as the scanning lines 5. When the on voltage is applied via the scanning lines 5, the TFTs 8 are turned on to release the electric charge accumulated in the radiation detection elements 7. When the off voltage is applied via the scanning lines 5, the TFTs 8 are turned off to break continuity between the radiation detection elements 7 and the signal lines 6, and accumulate in the radiation detection elements 7 the electric charge generated by the radiation detection elements 7.

A read IC 16 includes a plurality of read circuits 17. The read circuits 17 are connected to the signal lines 6. In the process of reading the image data D, when the radiation detection elements 7 release electric charge, the electric charge flows into the read circuits 17 via the signal lines 6, and amplification circuits 18 output voltage values according to the amount of the flowing electric charge. Correlated double sampling circuits (described as “CDSs” in FIG. 6) 19 read the voltage values output from the amplification circuits 18 as analog image data D, and outputs the same to the downstream side. The output image data D is sequentially transmitted to an A/D converter 20 via an analog multiplexer 21, converted sequentially by the A/D converter 20 into digital image data D, and output to a storage unit 23 and saved there in sequence.

A control unit 22 is composed of a computer in which a CPU, a ROM, a RAM, an input/output interface, and others not illustrated are connected to a bus, an FPGA (field programmable gate array), and the like. The control unit 22 may be composed of a dedicated control circuit. The control unit 22 is connected to a storage unit 23 composed of a SRAM (static RAM), an SDRAM (synchronous DRAM), an NAND-type flash memory, and the like.

The control unit 22 is also connected to a communication unit 30 that communicates with the outside via the antenna 29 and the connector 27 in a wireless or wired manner. The control unit 22 is further connected to a built-in power source 24 such as a lithium ion capacitor that supplies necessary power to the scanning drive unit 15, the read circuits 17, the storage unit 23, the bias power source 14, and others.

[About the Processes Performed in the Radiographic Image Shooting System at One-Shot Long Shooting]

Next, the processes performed in the radiographic image shooting system 50 according to the embodiment at the time of one-shot long shooting will be specifically described. In addition, the actions of the radiographic image shooting system 50 according to the embodiment will also be described.

As illustrated in FIG. 1, there is no particular problem in the radiographic image shooting system 50 in which the one shooting room Ra and the one console C are associated with each other in advance. As illustrated in FIG. 2, however, when a plurality of shooting rooms Ra (Ra1 to Ra3) and a plurality of consoles C are associated with one another via the network N or the like, the operator such as a radiographer, before shooting, specifies (or declares) the shooting room Ra in which the shooting stand 51A for one-shot long shooting is installed, on the console C to be used from now. When the shooting room Ra is specified on the console C in this manner, the console C and the specified shooting room Ra are associated with each other afterward.

Then, the operator such as a radiographer starts a necessary number of radiographic image shooting device F (that is, powers on the devices F or switches from the power consumption mode to the shooting mode in the devices F), and loads the radiographic image shooting devices F into the holder 51a of the shooting stand 51A. Specifically, three radiographic image shooting devices Fare loaded for long shooting of the entire lower extremity or the like of an adult patient, and about two radiographic image shooting devices F are loaded for long shooting of a child or infant patient. The radiographic image shooting devices F perform initial operations such as resetting the radiation detection elements 7.

[About the Connection of the Radiographic Image Shooting Devices to Dongles]

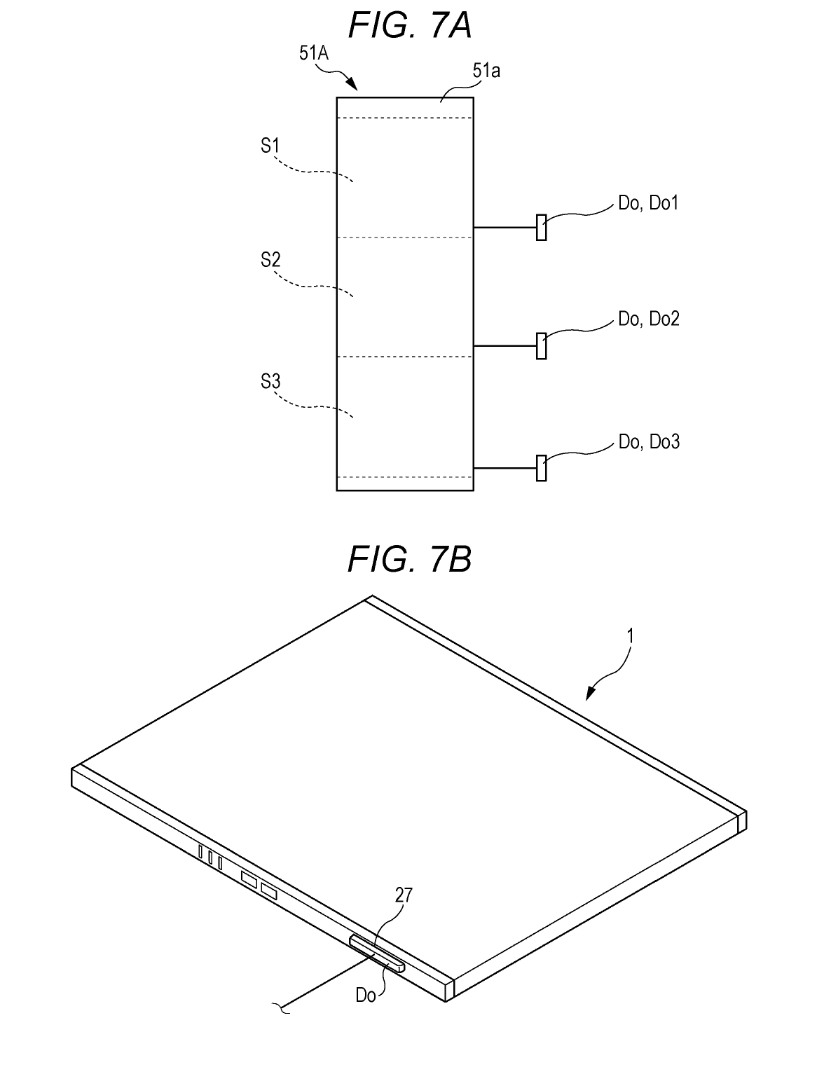

In the embodiment, as illustrated in FIG. 7A, the holder 51a of the shooting stand 51A has dongles Do1 to Do3 storing different identification information at loading positions S1 to S3 where the radiographic image shooting devices 1 can be loaded. In addition, as illustrated in FIG. 7B, the dongles Do are connected to the connectors 27 of the radiographic image shooting devices 1 before the loading of the radiographic image shooting devices 1 into the holder 51a.

Although not illustrated, for example, the dongles Do may be connected to USB terminals or the like of the radiographic image shooting devices 1. Instead of the operator such as a radiographer connecting manually the dongles Do to the connectors 27 of the radiographic image shooting devices 1, when the radiographic image shooting devices 1 are loaded into the holder 51a, the dongles Do corresponding to the loading positions may be automatically connected to the connectors 27 of the radiographic image shooting devices 1.

When the dongles Do are connected to the radiographic image shooting devices 1, the radiographic image shooting devices 1 read identification information stored in the dongles Do and send to the console C the same together with cassette IDs as their respective identification information. The console C has a correspondence table in which the identification information of the dongles Do1 to Do3 and the loading positions S1 to S3 in the holder 51a of the shooting stand 51A are associated with one another. When the radiographic image shooting devices 1 send the cassette IDs and the identification information of the dongles Do as described above, the console C refers to the correspondence table to recognize which of the radiographic image shooting devices 1 is loaded at which of the loading positions S1 to S3 in the holder 51a of the shooting stand 51A.

The radiographic image shooting devices 1 loaded in the holder 51a of the shooting stand 51A also transmit to the console C shooting parameters Pa such as the remaining amounts of their built-in power sources 24 (see FIG. 6) and radio field strengths for wireless communications with the access point 53 (see FIGS. 1 and 2), on a regular basis or upon receipt of a transmission request from the console C.

[About the Generation of a First Shooting Condition Key]

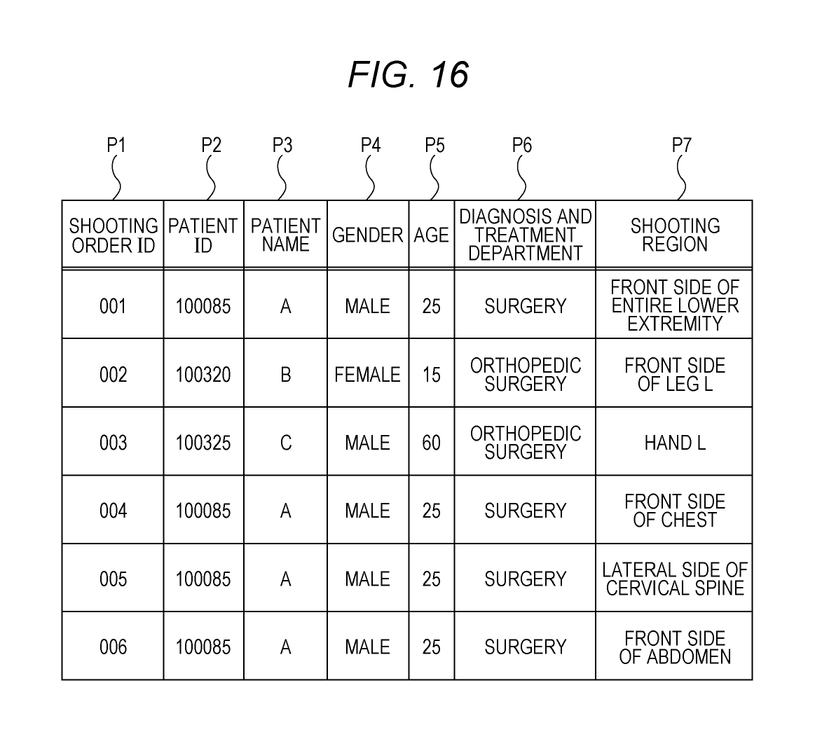

Before shooting, the console C obtains shooting order information regarding scheduled radiographic image shooting from the HIS and RIS described above, in response to the operation by the operator such as a radiographer. In the embodiment, the shooting order information includes “patient ID” P2, “patient's name” P3, “gender” P4, “age” P5, diagnosis and treatment department” P6, “shooting region” P7, and others, as shown in FIG. 16, for example. In the order shooting orders were accepted, “shooting order ID” P1 is automatically assigned to the shooting order information. As an item specified by the shooting order information, a modality to be additionally used (including the shooting stand 51A for one-shot long shooting) and the like may be specified. The items specified by the shooting order information can be decided as appropriate.

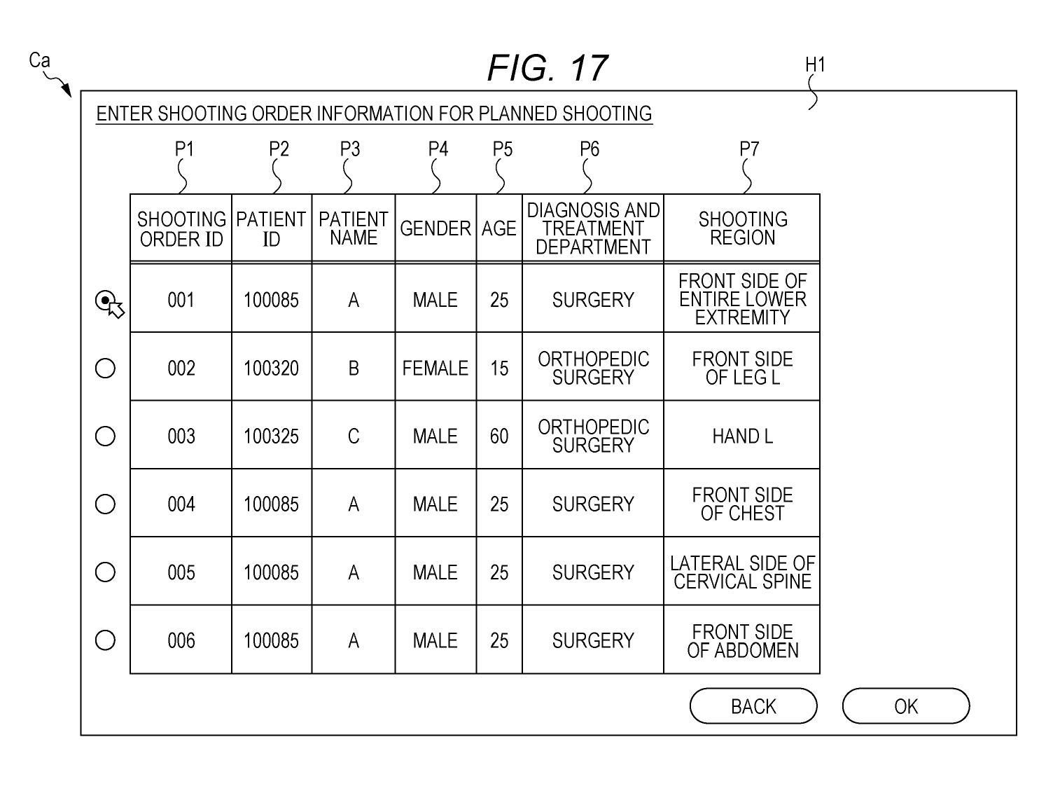

When obtaining the shooting order information, the console C displays on the display unit Ca a selection screen H1 including a list of the shooting order information as illustrated in FIG. 17 described above. Hereinafter, descriptions will be given as to the case where the shooting order information specifying the shooting region P7 as “entire lower extremity” (that is, long shooting) is selected on the selection screen H1.

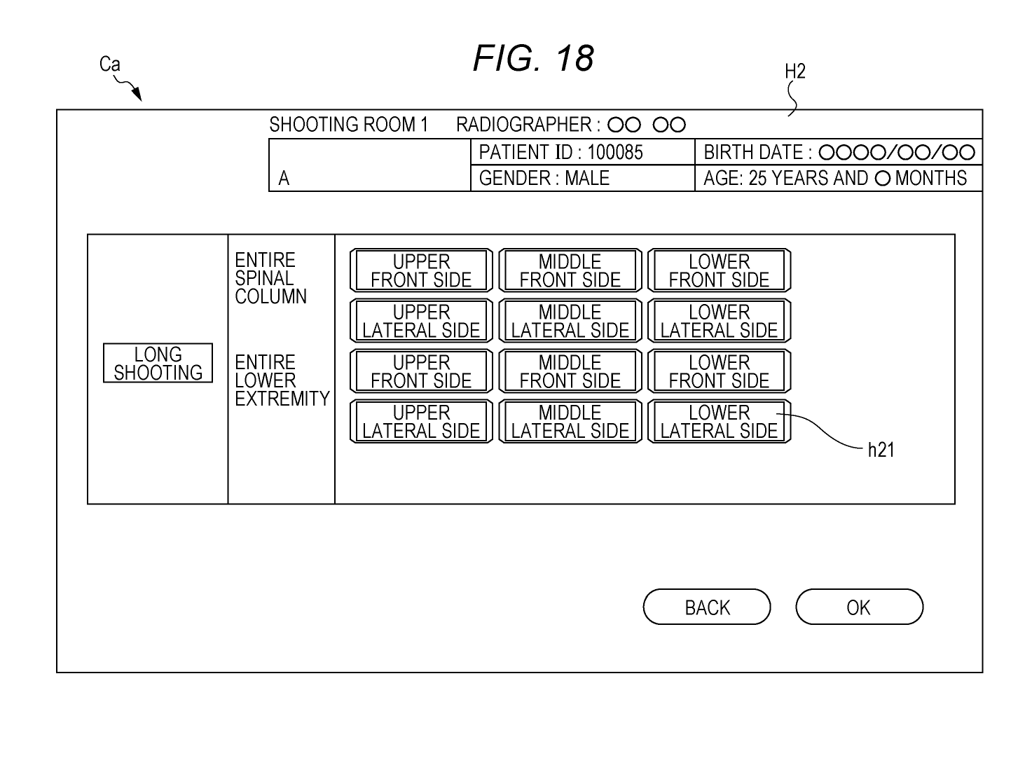

As described above, in the conventional example, when the shooting order information for long shooting is selected on the selection screen H1, the screen shifts to a setting screen H2 as illustrated in FIG. 18, and the operator such as a radiographer needs to set the shooting condition keys by clicking button icons h21 “upper front side (of entire lower extremity),” “middle front side,” “lower front side,” and others on the setting screen H2.

In the radiographic image shooting system 50 according to the embodiment, the operator such as a radiographer does not need to set the shooting condition keys but the console C creates automatically the shooting condition keys (a first shooting condition key K1 and second shooting condition keys K2). This will be specifically described below.

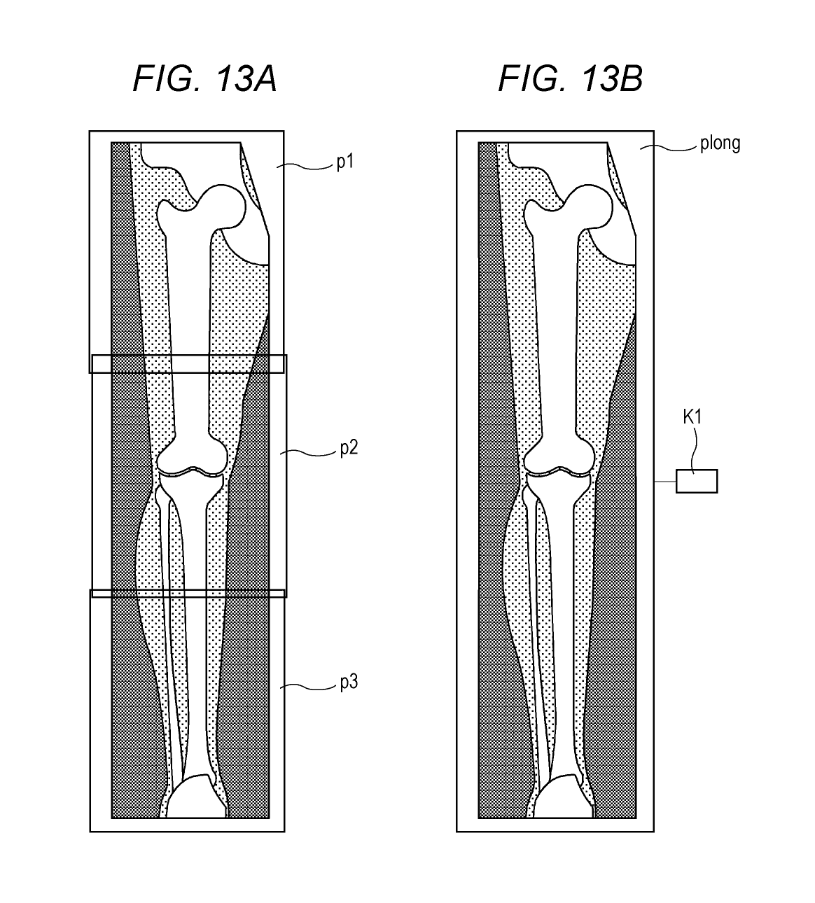

When the shooting order information for long shooting (that is, in this case, the shooting order information with the shooting region P7 specified as “front side of entire lower extremity”) is selected on the selection screen H1 (see FIG. 17) as described above, the console C creates the first shooting condition key K1 to be linked with a long image plong produced from a plurality of images (for example, see FIG. 13B and others described later) after long shooting, as described later.

As described above, the shooting order information is shooting information generated for each shooting (that is, one piece of information at one-time shooting), and the shooting condition key is shooting information generated for each image (that is, one key for one image). The shooting condition keys constitutes shooting information including the patient's name P3, the shooting region P7, and others as the shooting order information does, and the items for the shooting condition keys can be decided as appropriate.

[About the Start of the Irradiation Device]

When the shooting order information is selected on the console C as described above, the console C may transmit necessary information to the control unit 55 of the irradiation device 52 to start automatically the irradiation device 52. Alternatively, the operator such as a radiographer may operate manually the operator station 57 of the irradiation device 52 (see FIG. 1) to start the irradiation device 52.

[About how to Display on the Display Unit of the Console]

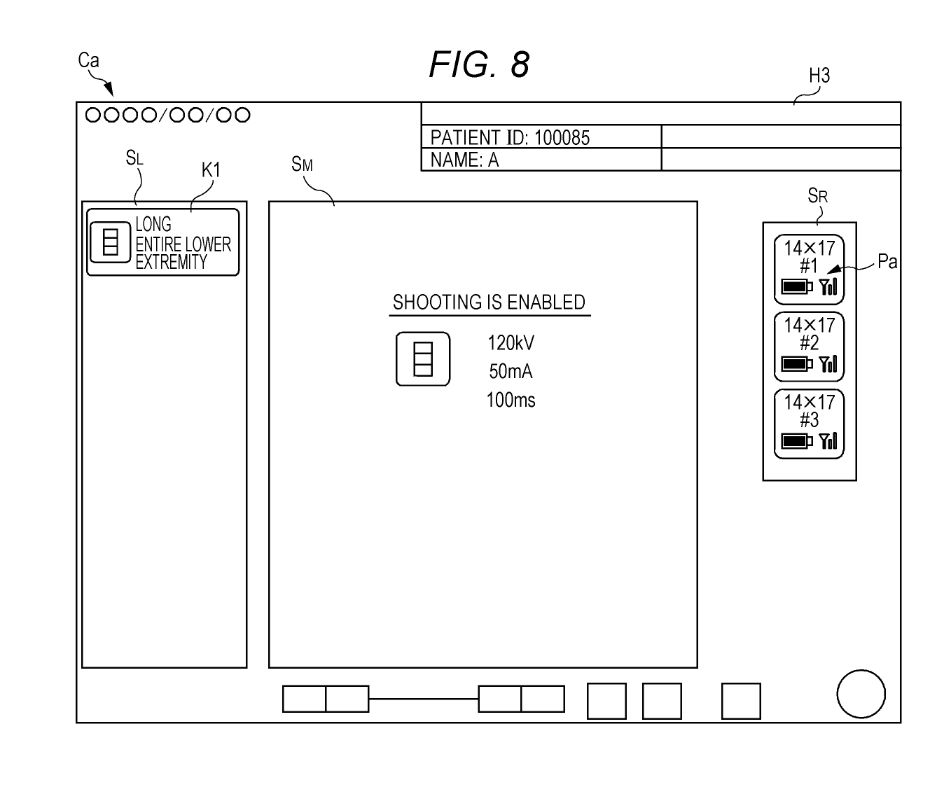

When the first shooting condition key K1 is created as described above, the console C switches the screen displayed on the display unit Ca from the selection screen H1 described above to a display screen H3 as illustrated in FIG. 8 (no transition takes place to the setting screen H2 as illustrated in FIG. 18). In the embodiment, the display screen H3 is provided with a central main screen S_{M} and left and right sub screens S_{L} and S_{R}.

The left sub screen S_{L} of the display screen H3 is a section where the selected shooting order information, that is, the shooting condition keys corresponding to individual shooting actions are displayed as simplified icons.

When the shooting order information for simple shooting (specifically, general shooting of the front side of the chest or the like by applying radiation once to the radiographic image shooting device 1 to produce one image) is selected on the selection screen H1 (see FIG. 17), for example, the console C creates the shooting condition key to be linked to the image produced by simple shooting, and displays the shooting condition key on the left sub screen S_{L} of the display screen H3, although not illustrated. In this case, the number of the produced image is one and therefore the number of the shooting condition key displayed as an icon on the sub screen S_{L} is also one.

When the first shooting condition key K1 is created as a result of selection of the shooting order information for long shooting on the selection screen H1 as described above, the console C displays the first shooting condition key K1 as a simplified icon on the left sub screen S_{L} of the display screen H3 as illustrated in FIG. 8, as in the case of the simple shooting.

In the case of long shooting, the plurality of radiographic image shooting devices 1 loaded in the holder 51a of the shooting stand 51A reads the image data D, and the console C produces a plurality of images p based on the image data D as described later. The console C creates second shooting condition keys K2 to be linked to the plurality of images p corresponding to the number of images p. However, the second shooting condition keys K2 are not displayed on the left sub screen S_{L} of the display screen H3.

That is, in the embodiment, both in the cases of simple shooting and long shooting, the number of the shooting condition key (the first shooting condition key in the case of long shooting) displayed on the left sub screen S_{L} of the display screen H3 is only one.

The second shooting condition keys K2 may be set by a radiographer or the like so as to be displayed on the left sub screen S_{L} of the display screen H3, as with the first shooting condition key K1. Nevertheless, at least in the default state (that is, in the initial setting state), only the first shooting condition key K1 is to be displayed on the left sub screen S_{L}.

As described above, in the embodiment, in the case of performing one-shot long shooting, only one corresponding first shooting condition key K1 is displayed on the left sub screen S_{L} of the display screen H3 but the second shooting condition keys K2 are not basically displayed on the left sub screen S_{L} of the display screen H3, as in the case of performing simple shooting. Accordingly, the operator such as a radiographer can perform long shooting with the operational feeling as if performing simple shooting.

Various kinds of information can be displayed and produced images can be displayed in enlarged sizes on the central main screen S_{M} of the display screen H3.

In the embodiment, before long shooting, the console C indicates on the main screen S_{M} whether long shooting is enabled. At that time, when all of the radiographic image shooting devices 1 for one-shot long shooting loaded in the holder 51a of the shooting stand 51A become capable of shooting, the console C notifies the operator such as a radiographer that long shooting is enabled by displaying the indication on the main screen S_{M} or the like. The notification may be made by sound or the like.

When any one of the radiographic image shooting devices 1 loaded in the holder 51a of the shooting stand 51A is incapable of shooting, the console C displays the message “in preparation,” “shooting is disabled,” or the like on the main screen S_{M} to notify the operator such as a radiographer that long shooting is disabled.

In the case of one-shot long shooting, when any one of the radiographic image shooting devices 1 loaded in the holder 51a of the shooting stand 51A is incapable of shooting, one-shot long shooting is not allowed. One-shot long shooting becomes enabled only when all of the radiographic image shooting devices 1 become capable of shooting.

By displaying simply the information indicating whether shooting is enabled or disabled on the main screen S_{M} of the display screen H3, not displaying the states and others of the radiographic image shooting devices 1 loaded in the holder 51a of the shooting stand 51A on the main screen S_{M} of the display screen H3, the operator such as a radiographer can perform long shooting with the operational feeling as if performing simple shooting.

There are various possible situations in which one-shot long shooting is disabled. For example, when no radiographic image shooting device 1 is loaded in the holder 51a of the shooting stand 51A, shooting is disabled. Accordingly, in such a case, the message “load panel” or the like is displayed on the main screen S_{M}, for example. The panel means the radiographic image shooting device 1. It can be decided as appropriate whether shooting is to be enabled or disabled when only one radiographic image shooting device 1 is loaded.

When the radiographic image shooting device 1 is loaded into the holder 51a of the shooting stand 51A but shooting preparations are not made because initial operations such as resetting of the radiation detection elements 7 is not yet completed or the like, for example, the message “in preparation,” “waiting,” or the like is displayed on the main screen S_{M}. In addition, as described above, shooting is also disabled when it is determined based on the shooting parameters Pa, such as the remaining amount of the built-in power source 24 (see FIG. 6) and radio field strength or the like for wireless communications with the access point 53 (see FIGS. 1 and 2), transmitted from the radiographic image shooting device 1 loaded in the holder 51a of the shooting stand 51A, that the radiographic image shooting device 1 has run out of battery or the radio field strength is too low to perform wireless communications. In the case where shooting is disabled as described above, the message “shooting is disabled” or the like is displayed on the main screen S_{M}, for example.

When a plurality of radiographic image shooting devices 1 is loaded in the holder 51a of the shooting stand 51A and there are the radiographic image shooting devices 1 at the loading positions S1 and S3 out of the loading positions S1 to S3 in the holder 51a illustrated in FIG. 7A but there is no radiographic image shooting device 1 at the intermediate loading position S2, for example, the console C may determine that shooting is disabled. In such a case, the message “shooting is disabled” or “load panel” may be displayed on the main screen S_{M}, for example.

When shooting is disabled, the operator such as a radiographer cannot take measures such as replacing the radiographic image shooting devices 1, changing the loading positions, or the like, unless it is known which of the radiographic image shooting devices 1 loaded in the holder 51a of the shooting stand 51A has run out of battery or is incapable of wireless communications, or the defective radiographic image shooting device 1 is loaded at which of the loading positions in the holder 51a.

In the embodiment, before shooting, the console C displays the shooting parameters Pa such as the loading positions S1 to S3 of the radiographic image shooting devices 1 loaded in the holder 51a of the shooting stand 51A, the remaining amount of the built-in power source 24, and the radio field strength for wireless communications with the access point 53, on the right sub screen S_{R} of the display screen H3, as illustrated in FIG. 8.

At that time, the console C recognizes which of the radiographic image shooting devices 1 is loaded at which of the loading positions S1 to S3 in the holder 51a of the shooting stand 51A, based on information such as the identification information provided by the radiographic image shooting devices 1 connected to the dongles Do (see FIGS. 7A and 7B) at the time of loading into the holder 51a. Accordingly, in the embodiment, the console C displays the shooting parameters Pa of the radiographic image shooting devices 1 as simplified icons on the upper and lower positions of the sub screen S_{R} corresponding to the loading positions S1 to S3 of the holder 51a, thereby to display the loading positions S1 to S3 and the shooting parameters Pa of the radiographic image shooting devices 1 at the same time.

When the radiographic image shooting devices 1 are loaded at the loading positions S1 and S3 in the holder 51a of the shooting stand 51A but no radiographic image shooting device 1 is loaded at the loading position S2, the shooting parameters Pa are displayed as icons in the upper and lower display portions, out of the upper, middle, and lower display portions of the sub screen S_{R} of the display screen H3, but no shooting parameters Pa are displayed at the middle display portion. Accordingly, the operator such as a radiographer can see the display alone on the sub screen S_{R} to recognize not only the shooting parameters Pa of the radiographic image shooting devices 1 but also the loading positions S1 to S3 of the radiographic image shooting devices 1. Therefore, by providing the display described above on the sub screen S_{R}, the console C displays on the display screen H3 the loading positions S1 to S3 of the radiographic image shooting devices 1 loaded in the holder 51a of the shooting stand 51A.

According to this configuration, when shooting is disabled, the operator such as a radiographer can see the right sub screen S_{R} of the display screen H3 to easily recognize the presence or absence of the radiographic image shooting devices 1 loaded at the loading positions S1 to S3 in the holder 51a of the shooting stand 51A, which of the radiographic image shooting devices 1 has run out of battery or is incapable of wireless communications, or the like. Accordingly, the operator such as a radiographer can take appropriate measures such as loading the radiographic image shooting device 1 having not been yet loaded, changing the loading positions of the radiographic image shooting devices 1, or replacing the radiographic image shooting device 1.

As for the shooting parameters Pa of the radiographic image shooting devices 1, when the radiographic image shooting devices 1 are connected to the dongles Do to transmit to the console C the parameters Pa such as the remaining amount of the built-in power source 24 and the radio field strength for wireless communications with the access point 53, the shooting parameters Pa are displayed on the right sub screen S_{R} of the display screen H3. Each time the radiographic image shooting devices 1 transmit the parameters Pa on a regular basis or upon receipt of a transmission request from the console C, the parameters Pa on the sub screen S_{R} are updated.

To display the shooting parameters Pa of the radiographic image shooting devices 1, for example, the parameters Pa enabling shooting may be displayed in green or the like, the parameters Pa disabling shooting may be displayed in red, yellow, or the like, so that the operator such as a radiographer can recognize easily the parameters Pa disabling shooting.

[About Inhibiting the Emission of Radiation when Shooting is Disabled]

Even though shooting is disabled, the operator such as a radiographer may press fully the exposure switch 56 of the irradiation device 52 (see FIG. 4C) to emit radiation from the irradiation device 52. In this case, the exposure is not useless and the exposure dose given to the patient increases to put larger burden on the patient's body.

Accordingly, the console C may permit the emission of radiation from the irradiation device 52 only when all the radiographic image shooting devices 1 loaded in the holder 51a of the shooting stand 51A are capable of shooting, and may inhibit the emission of radiation from the irradiation device 52 when any one of the radiographic image shooting devices 1 is incapable of shooting.

Specifically, as described above, when the console C determines that no radiographic image shooting device 1 is loaded in the holder 51a of the shooting stand 51A or that preparations for shooting by the loaded radiographic image shooting devices 1 are not yet made, or when shooting is not allowed because the radiographic image shooting device 1 has run out of battery or the radio field strength is too low, even though the operator such as a radiographer presses fully the exposure switch 56 of the irradiation device 52 and the control unit 55 of the irradiation device 52 transmits a radiation start signal as described later, for example, no interlock release signal is transmitted from the console C to the irradiation device 52 to inhibit the emission of radiation from the irradiation device 52.

Then, only when all the radiographic image shooting devices 1 loaded in the holder 51a of the shooting stand 51A are capable of shooting, the console C may transmit an interlock release signal to the irradiation device 52 to permit the emission of radiation from the irradiation device 52.

According to this configuration, it is possible to prevent accurately a problem that, even though shooting is disabled, the irradiation device 52 emits radiation to waste exposure and put a larger burden on the patient's body due to an increased exposure dose.

[About the Notification that Shooting is Enabled and the Emission of Radiation from the Irradiation Device]

Meanwhile, when determining that all the radiographic image shooting devices 1 loaded in the holder 51a of the shooting stand 51A are capable of shooting, the console C notifies the operator such as a radiographer that one-shot long shooting is enabled by displaying the message “display is enabled” or the like on the main screen S_{M} of the display screen H3 as illustrated in FIG. 8.

The operator such as a radiographer completes the positioning of the patient P as a subject and the holder 51a of the shooting stand 51A (that is, the radiographic image shooting devices 1) in the shooting room Ra (see FIGS. 1 and 2), returns to the front room Rb to check the notification that shooting is enabled displayed on the display screen H3 of the console C, and then operates the exposure switch 56 to emit radiation from the irradiation device 52.

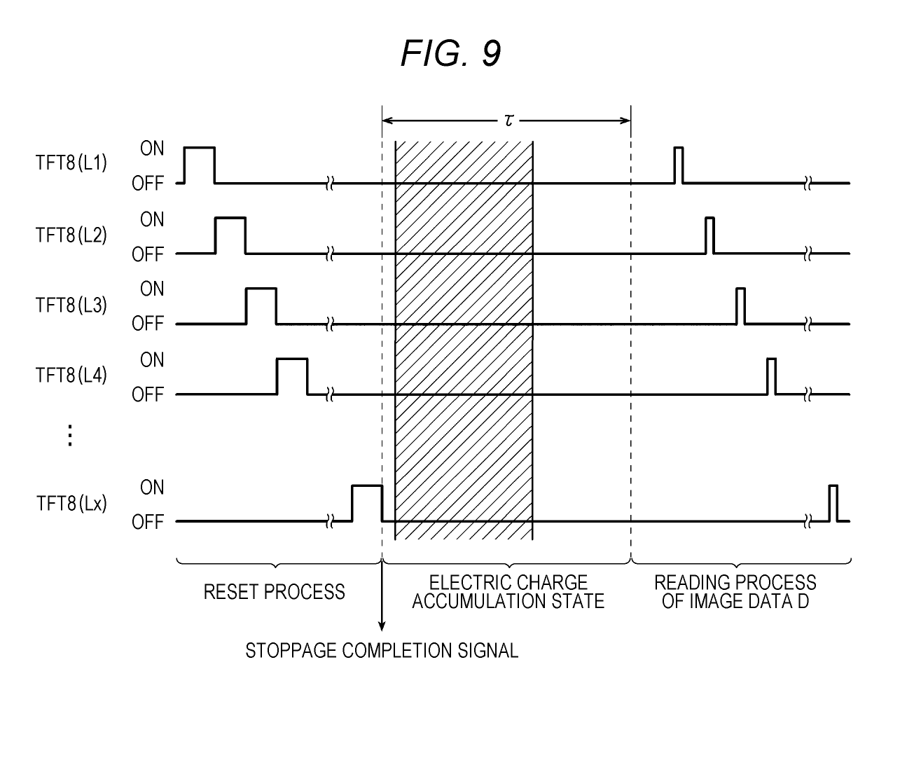

At that time, as illustrated in FIG. 9, for example, the radiographic image shooting devices 1 loaded in the holder 51a of the shooting stand 51A apply the on voltage sequentially from the gate drivers 15b of the scanning drive unit 15 (see FIG. 6) to the lines L1 to Lx of the scanning lines 5 to reset the radiation detection elements 7 with removal of electric charge from the radiation detection elements 7.

Then, the operator such as a radiographer presses fully the exposure switch 56 to transmit an irradiation start signal to the console C from the control unit 55 of the irradiation device 52 via the relay 54 and the access point 53, and the console C transmits to the radiographic image shooting devices 1 a signal for instructing stoppage of the reset process of the radiation detection elements 7.

The radiographic image shooting devices 1 stop the reset process of the radiation detection elements 7 when the application of the on voltage to the last line Lx of the scanning lines 5 is completed. After the stoppage of the reset process of the radiation detection elements 7, the radiographic image shooting devices 1 transmit a stoppage completion signal to the console C and apply the off voltage from the gate drivers 15b to the lines L1 to Lx of the scanning lines 5 to shift to the electric charge accumulation state in which the electric charge generated in the radiation detection elements 7 by radiation is accumulated in the radiation detection elements 7.

Upon receipt of the stoppage completion signals from all the radiographic image shooting devices 1 loaded in the holder 51a of the shooting stand 51A, the console C transmits an interlock release signal to the control unit 55 of the irradiation device 52. The control unit 55 of the irradiation device 52 emits radiation from the irradiation device 52 only when receiving the interlock release signal.

In the embodiment, long shooting is performed by one-shot radiation from the irradiation device 52 to the radiographic image shooting devices 1 loaded in the holder 51a of the shooting stand 51A in such a manner described above.

In the embodiment, as described above, the irradiation device 52 and the console C (and the radiographic image shooting devices 1) perform long shooting in cooperation with each other (that is, by a cooperation method) while exchanging signals and others. Alternatively, the irradiation device 52 and the radiographic image shooting devices 1 may perform long shooting without exchanging signals and others (that is, by a non-cooperation method). In that case, the radiographic image shooting devices 1 detect by themselves the emission of radiation from the irradiation device 52 and shift to the electrical charge accumulation state. For the detail of a method for the radiographic image shooting devices 1 to detect the emission of radiation and others, refer to JP 2009-219538 A, WO 2011/135917, WO 2011/152093, and others, for example.

[About the Creation of the Second Shooting Condition Keys]

In the embodiment, when the radiographic image shooting device 1 is loaded in the holder 51a of the shooting stand 51A, one of the dongles Do1 to Do3 corresponding to the loading positions S1 to S3 is connected to the connector 27 of the radiographic image shooting device 1. Then, the radiographic image shooting device 1 connected to the dongle Do sends to the console C the identification information and the cassette ID, the console C detects which of the radiographic image shooting devices 1 is loaded at which of the loading positions S1 to S3 in the holder 51a of the shooting stand 51A.

That is, in the embodiment, the console C serves as a detection unit that detects the presence or absence of the loaded radiographic image shooting device 1 in the holder 51a of the shooting stand 51A and the loading positions S1 to S3. The detection unit may be provided separately from the console C, and the dongles Do or the like may have the function of the detection unit.

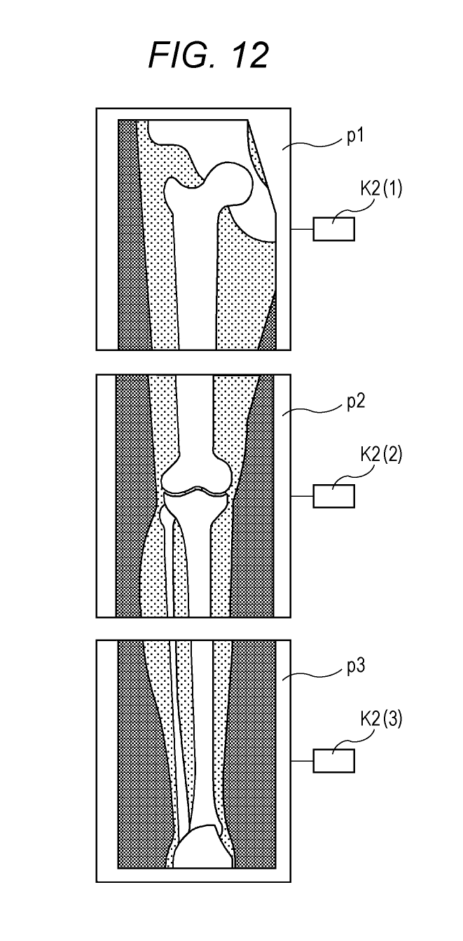

In the present invention, the console C determines the number N (three in FIG. 12) of images P (see p1 to p3 in FIG. 12 described later, for example) for use in creation of a long image plong (for example, see FIG. 13B and others described later, for example), and newly creates a number of second shooting condition keys corresponding to the determined number N of the images p, in association with the loading position of the radiographic image shooting devices 1 having shot the images p in the holder 51a of the shooting stand 51A.

In the embodiment, the console C serves as the detection unit described above. Taking advantage of this, the console C detects the presence or absence of the radiographic image shooting devices 1 loaded in the holder 51a of the shooting stand 51A and the loading positions S1 to S3, and determines the number n of the radiographic image shooting devices 1 loaded in the holder 51a of the shooting stand 51A at the time of emission of radiation from the irradiation device 52, based on the results of the detection. The console C then determines the number N (N is equal to n) of the images p for use in the creation of the long image plong based on the determined number n of the radiographic image shooting devices 1, and creates newly the N second shooting condition keys K2 corresponding to the number N of the images p in association with the loading positions of the radiographic image shooting devices 1.

Specifically, as described above, in some cases, the operator such as a radiographer initially loads two radiographic image shooting devices 1 in the holder 51a of the shooting stand 51A, for example, but later may determine that one more radiographic image shooting device 1 is necessary and load newly a third radiographic image shooting device 1. In other cases, the operator such as a radiographer initially loads three radiographic image shooting devices 1, but later may find out that one of them is defective or determine that only two are sufficient or the like, and then remove one of the radiographic image shooting devices 1 and perform long shooting by the remaining two radiographic image shooting devices 1.

In this manner, the number n of the radiographic image shooting devices 1 loaded in the holder 51a of the shooting stand 51A may change. In the embodiment, when the exposure switch 56 is fully pressed to transmit a radiation start signal to the console C or when the console C transmits an interlock release signal to the irradiation device 52 as described above, for example, the console C can recognize the emission of radiation from the irradiation device 52. Then, based on the identification information on the dongles Do and others transmitted from the radiographic image shooting devices 1 connected to the dongles Do, the console C can determine the number n of the radiographic image shooting devices 1 loaded in the holder 51a of the shooting stand 51A at that time (that is, the number N of the images p for use in the creation of the long image plong, which is also applied to the following descriptions).

Accordingly, the console C creates the second shooting condition keys K2 corresponding to the number n of the radiographic image shooting devices 1 identified in this manner (that is, the number n of the radiographic image shooting devices 1 loaded in the holder 51a at the time of emission of radiation from the irradiation device 52).

At that time, the console C associates the second shooting condition keys K2 with the corresponding loading positions of the radiographic image shooting devices 1. When three radiographic image shooting devices 1 are loaded, for example, the console C creates the three second shooting condition keys K2 for the segmented parts of the shooting region P7 “upper front side of entire lower extremity,” “middle front side of entire lower extremity,” and “lower front side of entire lower extremity.” In the following descriptions, the second shooting condition keys K2 for the segmented parts of the shooting region P7 “upper front side of entire lower extremity,” “middle front side of entire lower extremity,” and “lower front side of entire lower extremity” will be referred to as the second shooting condition keys K2(1), K2(2), and K2(3).

When two radiographic image shooting devices 1 are loaded, for example, the console C creates two second shooting condition keys K2 for the segmented parts of the shooting region P7 “upper front side of entire lower extremity” and “middle front side of entire lower extremity,” or “middle front side of entire lower extremity” and “lower front side of entire lower extremity,” in the second shooting condition keys K2 depending on the loading positions of the radiographic image shooting devices 1.

In this manner, when the irradiation device 52 emits radiation, the console C automatically creates the n second shooting condition keys K2 corresponding to the number n of the radiographic image shooting devices 1 loaded in the holder 51a of the shooting stand 51A at the time of emission of radiation from the irradiation device 52, in association with the loading positions (upper, middle, and lower positions) of the radiographic image shooting devices 1.

In the embodiment, the console C does not basically display the created second shooting condition keys K2 on the left sub screen S_{L} of the display screen H3 but displays the first shooting condition key K1 on the sub screen S_{L}.

[About Reading, Transfer, and Others of Image Data and Offset Data]

When the radiographic image shooting devices 1 loaded in the holder 51a of the shooting stand 51A shift to the electric charge accumulation state and the irradiation device 52 emits radiation to perform one-shot long shooting as illustrated in FIG. 9 (the shaded section in FIG. 9 means the emission of radiation), the control unit 22 of the radiographic image shooting devices 1 apply sequentially the on voltage from the gate drivers 15b to the lines L1 to Lx of the scanning lines 5 for the reading process of the image data D described above.

The control unit 22 of the radiographic image shooting devices 1 read the image data D and transfer the same to the console C via the antenna 29 in a wireless method. That is, when three radiographic image shooting devices 1 are loaded in the holder 51a of the shooting stand 51A, for example, the three radiographic image shooting devices 1 transfer the image data D to the console C concurrently. Instead of transferring the image data D, the radiographic image shooting devices 1 may extract preview image data Dp to be displayed on the display screen H3 from the image data D and transfer ahead of the image data D as described later.

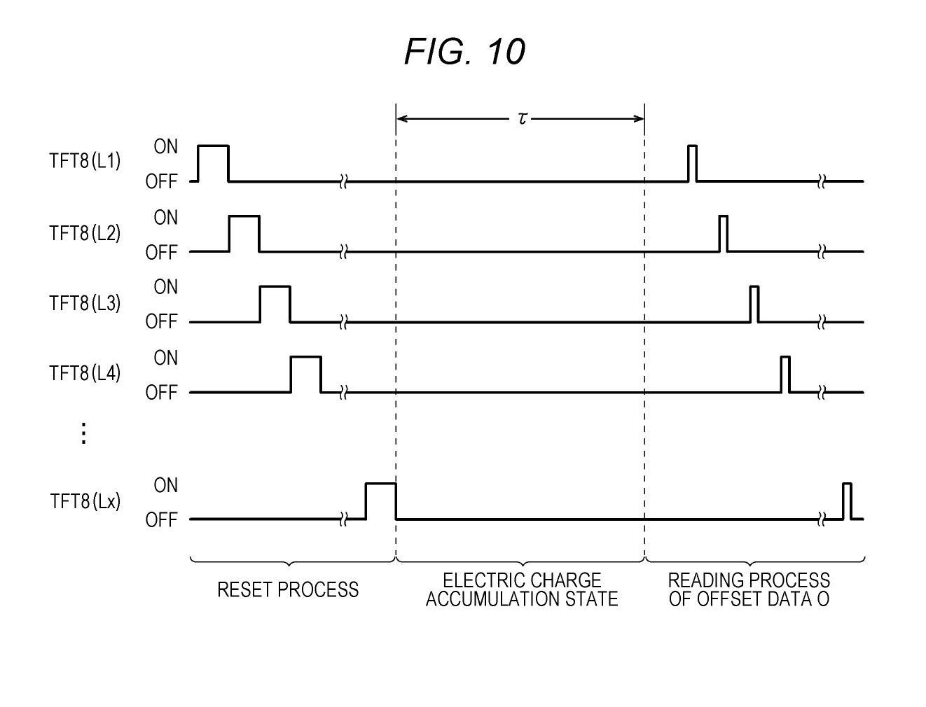

The radiographic image shooting devices 1 perform the reading process of offset data O as illustrated in FIG. 10 concurrently with the transfer of the image data D and the preview image data. Specifically, upon completion of the reading process of the image data D as described above, the radiographic image shooting devices 1 then resets the radiation detection elements 7 for one or predetermined number of frames and shift to the electric charge accumulation state as illustrated in the left part of FIG. 10. Then, when no radiation is emitted to the radiographic image shooting devices 1, the radiographic image shooting devices 1 are continuously kept in the electric charge accumulation state for the same period of time as duration T of the electric charge accumulation state before the reading of the image data D, and then apply sequentially the on voltage from the gate drivers 15b to the lines L1 to Lx of the scanning lines 5 and read the offset data O from the radiation detection elements 7 in the same manner as the reading process of the image data. The reading process of the offset data O may be performed prior to long shooting.

After reading the offset data O as described above, when having already transferred the preview image data Dp, the radiographic image shooting devices 1 transfer to the console C the remaining image data D and the offset data O. When the preview image data Dp is not to be extracted, the radiographic image shooting devices 1 transfer to the console C the offset data O subsequent to the transfer of the image data D having been already started.

[About Display of a Preview Image]

When the image data D or the preview image data Dp (hereinafter, the image data D will be described as a representative, but the following descriptions are also applied to the preview image data Dp) has been transferred but the offset data O has not yet been transferred, the console C subtracts tentative offset data Op from the image data D at each transfer of the image data D to calculate a subtracted value Dp* for each of the radiation detection elements 7 of the radiographic image shooting devices 1 according to the following equation (1):

Dp*=D−Op (1)

The console C performs simple image processing on the values Dp* and displays preview images p_pre at the positions corresponding to the radiographic image shooting devices 1 on the right sub screen S_{R} of the display screen H3 (that is, the positions where the shooting parameters Pa of the radiographic image shooting devices 1 are displayed as icons). Accordingly, the preview images p_pre are displayed in a wipe form (that is, the displayed portions increase from the upper to lower sides, for example) at the positions corresponding to the radiographic image shooting devices 1 on the right sub screen S_{R} of the display screen H3.

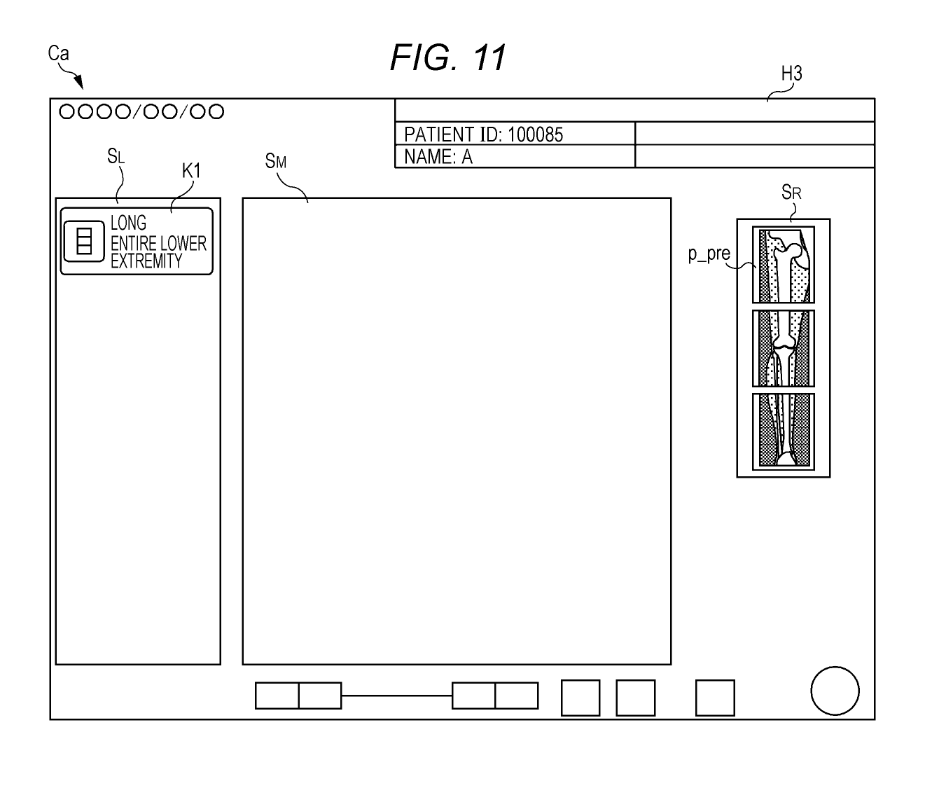

Upon completion of the transfer of the image data D from the radiographic image shooting devices 1, the preview images p_pre are displayed at the positions corresponding to the radiographic image shooting devices 1 on the right sub screen S_{R} of the display screen H3 as illustrated in FIG. 11, for example. Then, the operator such as a radiographer checks these preview images p_pre and determines whether re-shooting is necessary.

[About the Production of Images and Linkage with the Second Shooting Condition Keys]

Upon receipt of the image data D and the offset data O from the radiographic image shooting devices 1, the console C subtracts the offset data O from the image data D for each of the radiation detection elements 7 of the radiographic image shooting devices 1 according to the following equation (2) to calculate true image data D*:

D*=D−O (2)

The console C then performs accurate image processing on the calculated true image data D* such as gain correction, defective pixel correction, and gradation processing depending on the shooting regions for each of the radiographic image shooting devices 1 to produce images p1 to p3. The console C then links the produced images p1, p2, and p3 with the second shooting condition keys K2(1), K2(2), and K2(3) created as described above, respectively, as illustrated in an imaginary manner in FIG. 12.

Specifically, the console C links the images p1 to p3 produced based on the image data D or the like acquired from the radiographic image shooting devices 1 with the second shooting condition keys K2(1) to K2(3) corresponding to the loading positions S1 to S3 of the radiographic image shooting devices 1, respectively, by writing the second shooting condition key K2(1) into the header of the data of the produced image p1 or the like.

In the embodiment, when the irradiation device 52 emits radiation as described above, the console C determines the number n of the radiographic image shooting devices 1 loaded in the holder 51a of the shooting stand 51A, that is, the number N (three in the foregoing case) of the images p for use in the production of the long image plong. According to the determined number N of the images p, the console C automatically links the second shooting condition keys K2(1) to K2(3) automatically created in association with the loading positions S1 to S3 of the radiographic image shooting devices 1, with the images p1 to p3 acquired by the radiographic image shooting devices 1.

Accordingly, in the embodiment, the console C can create a number of second shooting condition keys K2 corresponding to the number of the radiographic image shooting devices 1 loaded in the holder 51a at the time of emission of radiation, and automatically and accurately link the second shooting condition keys K2 with the produced images p, without causing the operator such as a radiographer to create the second shooting condition keys K2, if anything, without the operator's knowledge.

[About the Production of a Long Image and Linkage with the First Shooting Condition Key]

Subsequently, the console C aligns the ends of the images p1 to p3 as illustrated in FIG. 13A and composites the images p1 to p3 to the long image plong as illustrated in FIG. 13B. The images p1 to p3 can be aligned and composited by the publicly-known method described in JP 2013-154146 A or the like, for example.

In the embodiment, the console C links the composite long image plong with the first shooting condition key K1 described above, as illustrated in FIG. 13B.

As described above, in the radiographic image shooting system 50 according to the embodiment, the operator such as a radiographer does not need to create the first shooting condition key K1 to be linked with the long image plong or the second shooting condition keys K2 to be linked with the images p taken by the radiographic image shooting devices 1, or does not need to link the first shooting condition key or the second shooting condition keys K2 with the long image plong or the images p. The console C automatically performs all these processes. Accordingly, the operator such as a radiographer can perform long shooting with the operational feeling as if performing simple shooting, without having to consider the number n of the radiographic image shooting devices 1 used for the shooting (that is, the number N of the images p for production of the long image plong).

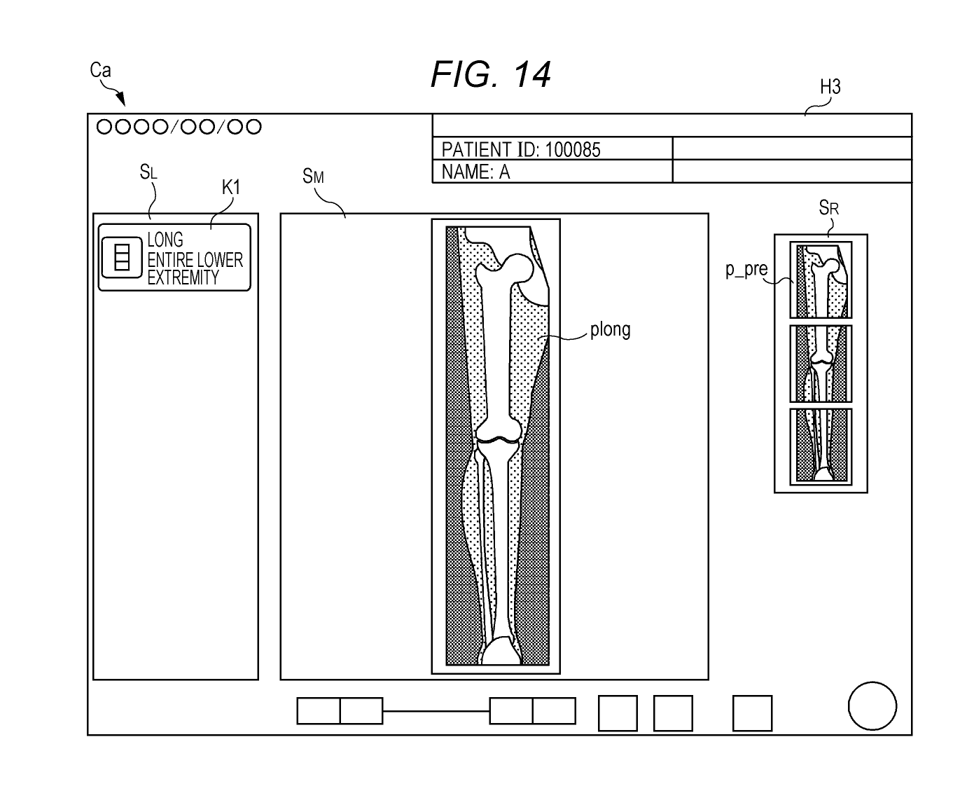

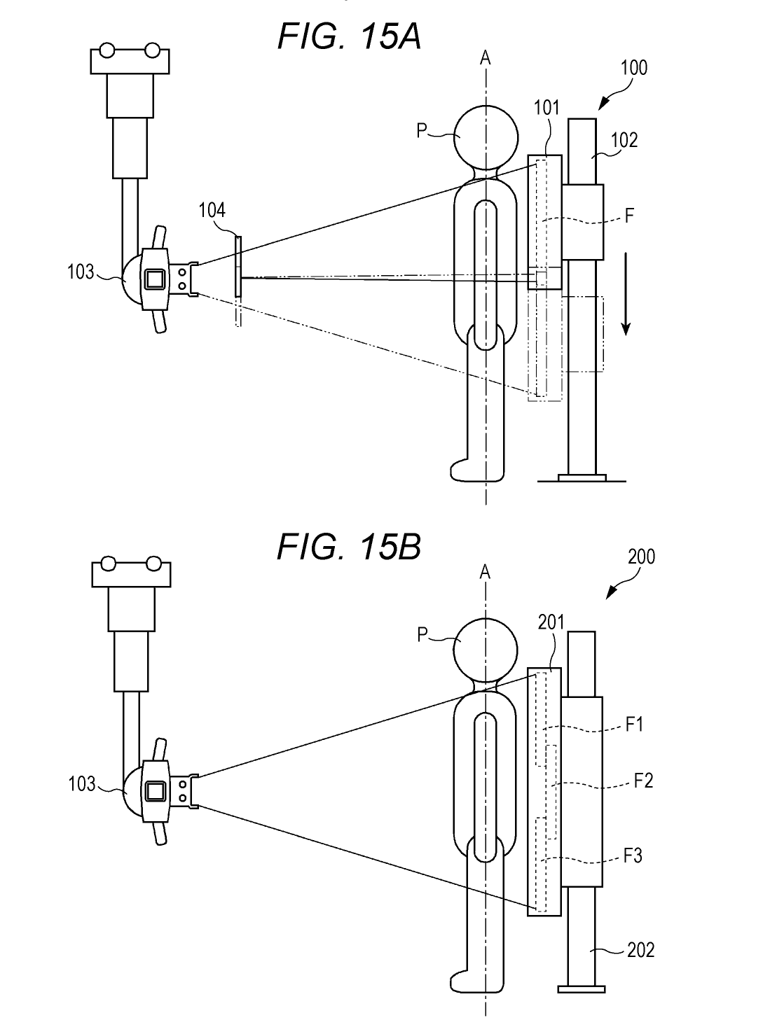

The console C displays the produced long image plong on the main screen S_{M} of the display screen H3 as illustrated in FIG. 14. Then, when the operator such as a radiographer checks and confirms the displayed long image plong, the console C associates the long image plong (see FIG. 13B) linked with the first shooting condition key K1 as described above, with the images p1 to p3 (see FIG. 12) linked with the second shooting condition keys K2(1) to K2(3), respectively.

Specifically, the console C associates the long image plong with the images p1 to p3 by writing common identification numbers or the like to the header of the data of the long image plong in which the first shooting condition key K1 is written and the headers of the data of the images p1 to p3 in which the second shooting condition keys K2(1) to K2(3) are written, for example.

According to this configuration, it is possible to manage integrally as one group the long image plong and its constituent images p1 to p3 together with the first shooting condition key K1 and the second shooting condition keys K2(1) to K2(3).

Timings for the linking of the second shooting condition keys K2 with the images p, the linking of the first shooting condition key K1 with the long image plong, the confirmation of the long image plong, and the association of the long image plong and the images p, orders of these processes, and the like can be decided as appropriate. That is, the foregoing processes may be performed in sequence as described above, for example. Alternatively, the linking processes and the association process may be performed after the operator such as a radiographer has performed the confirmation process of the long image plong.

Upon completion of the foregoing processes, the console C outputs the long image plong and the images p1 to p3 (including the first shooting condition key K1 and the second shooting condition keys K2) to an external system such as PACS, and then terminates the one-shot long shooting.

### ADVANTAGEOUS EFFECT

According to the radiographic image shooting system 50 of the embodiment as described above, at one-shot long shooting, the operator such as a radiographer does not create shooting condition keys to be linked with the long image plong and its constituent images p, but the console C automatically creates the first shooting condition key K1 to be linked with the long image plong. In addition, when the irradiation device 52 emits radiation, for example, the console C determines the n of the radiographic image shooting devices 1 loaded in the holder 51a of the shooting stand 51A to determine the number N of the images p for use in the production of the long image plong, creates automatically a number of new second shooting condition keys K2 corresponding to the determined number N of the images p in association with the loading positions S1 to S3 (that is, upper, middle, and lower positions, or the like), and links automatically the images p acquired from the radiographic image shooting devices 1 loaded in the holder 51a with the corresponding second shooting condition keys K2. The console C also links automatically the long image plong produced by compositing the images p with the first shooting condition key K1.

Accordingly, the operator such as a radiographer does not need to create the first shooting condition key K1 to be linked with the long image plong or the second shooting condition keys K2 to be linked with the images p taken by the radiographic image shooting devices 1, or does not need to link the first shooting condition key or the second shooting condition keys K2 with the long image plong or the images p. The console C performs automatically all the processes, and thus the operator such as a radiographer does not have to consider the number n of the radiographic image shooting devices 1 used for shooting.

In simple shooting, the number of the radiographic image shooting device 1 used for shooting is one. Accordingly, the operator such as a radiographer does not have to consider the number of the radiographic image shooting device 1. In addition, the console C creates automatically the shooting condition keys, and thus the operator does not need to create the shooting condition keys.

Therefore, according to the radiographic image shooting system 50 of the embodiment, the operator such as a radiographer can perform one-shot long shooting with the operational feeling as if performing simple shooting.

As in modification examples described later, in the embodiment described above, the first shooting condition key K1 and the second shooting condition keys K2 are linked with the long image plong and the images p by writing these shooting condition keys into the headers of the data of the long image plong and the images p. However, the present invention is not limited to this but the linkage may be made by any other method by which the first shooting condition key K1 and the second shooting condition keys K2 can be properly linked with the long image plong and the images p.

The shooting stand 51A for one-shot long shooting may also be used for simple shooting by loading one radiographic image shooting device 1 to the holder 51a of the shooting stand 51A for one-shot shooting illustrated in FIGS. 1, 3, and others, or bringing one of the radiographic image shooting devices 1 loaded in the holder 51a into the state of being capable of shooting (keeping the other radiographic image shooting devices 1 in the sleep state), or the like.

As described above, the present invention is also applicable to the case where the shooting stand 51A for one-shot long shooting is intended for decubitus shooting. In that case, for example, the holder 51a of the shooting stand 51A may be made rotatable or turnable such that the shooting stand 51A for one-shot long shooting can be used both for upright shooting and decubitus shooting.

### Modification Example 1

In the foregoing embodiment, the operator such as a radiographer initially loads three radiographic image shooting devices 1, but later finds one of them to be defective or only two to be enough, and pulls one of the radiographic image shooting devices 1 out of the holder 51a of the shooting stand 51A, and performs long shooting by the two radiographic image shooting devices 1.

In some cases, however, when the user may power off the unused radiographic image shooting device 1 or switch its power mode to a power-saving mode (also called sleep mode or the like) without pulling the unused radiographic image shooting device 1 out of the holder 51a of the shooting stand 51A, for example, the radiographic image shooting device 1 may become unusable for shooting.

Accordingly, when the radiographic image shooting devices 1 loaded in the holder 51a of the shooting stand 51A are powered off or switched to the power-saving mode, for example, the radiographic image shooting devices 1 notify that to the console C. Specifically, the console C serves not only as a detection unit that detects the presence or absence of the radiographic image shooting devices 1 loaded in the holder 51a of the shooting stand 51A and the loading positions S1 to S3 as described above but also as a detection unit that detects whether the radiographic image shooting devices 1 are to be used for shooting.

Based on the results of the detection, the console C may determine the number n* of the radiographic image shooting devices 1 in the operable state out of the radiographic image shooting devices 1 loaded in the holder 51a of the shooting stand 51A when the irradiation device 52 emits radiation (that is, the radiographic image shooting devices 1 powered on or in the shooting mode), and based on the determined number n* of the radiographic image shooting devices 1, the console C may determine the number N (N is equal to n*) of the images p for use in the generation of the long image plong.

### Modification Example 2

In the foregoing embodiment and modification example 1, the console C detects the loading positions S1 to S3 (see FIG. 7A) of the radiographic image shooting devices 1 loaded in the holder 51a of the shooting stand 51A. Instead of this, the radiographic image shooting devices 1 may detect by themselves the loading positions S1 to S3 in the holder 51a of the shooting stand 51A.

In this case, as illustrated in FIGS. 7A and 7B, for example, when the radiographic image shooting devices 1 are loaded in the holder 51a of the shooting stand 51A, the connectors 27 of the radiographic image shooting devices 1 are connected to the dongles Do to read the identification information stored in the dongles Do, and store temporarily the same in their storage units 23 (see FIG. 6), or RAMs, or the like. At that time, as in the embodiment, the radiographic image shooting devices 1 may notify by themselves their respective cassette IDs and identification information of the dongles Do to the console C.

For example, when transferring the image data D and others to the console C after shooting, the radiographic image shooting devices 1 write the identification information of the dongles Do into the headers of the image data D or the like, so that the information on the detected loading positions (the identification information of the dongles Do) can be transmitted to the console C. Alternatively, the radiographic image shooting devices 1 (or the radiographic image shooting devices 1 also) may have the correspondence table described above to determine by themselves the loading positions S1 to S3 with reference to the correspondence table based on the identification information of the dongles Do read from the dongles Do, and transmit to the console C the information on the loading positions accompanied with the image data D.

In this case, the console C determines the number N of the images p for use in the production of the long image plong based on the number of the images p transferred and acquired from the radiographic image shooting devices 1 as described above. Besides, at the time of acquisition of the image data D and the image data D from the radiographic image shooting devices 1, the console C may acquire the information on the loading positions detected by the radiographic image shooting devices 1 together with the images p, and determine the loading positions S1 to S3 of the radiographic image shooting devices 1 loaded in the holder 51a of the shooting stand 51A based on the acquired information on the loading positions.

### Modification Example 3

As in the foregoing modification example 2, the console C determines the number N of the images p for use in the production of the long image plong based on the number of the images p acquired from the radiographic image shooting devices 1. Alternatively, neither the console C nor the radiographic image shooting devices 1 may detect the loading positions of the radiographic image shooting devices 1 in the holder 51a of the shooting stand 51A but the console C may subject the images p acquired from the radiographic image shooting devices 1 to image analysis to determine the loading positions of the radiographic image shooting devices 1 having taken the images p in the holder 51a.

In this case, when the number N of the images p acquired from the radiographic image shooting devices 1 is two, for example, the console C can determine that two radiographic image shooting devices 1 are loaded in the holder 51a of the shooting stand 51A or two of the loaded radiographic image shooting devices 1 are capable of shooting.

In this case, it is not easy to determine only by image analysis that the two radiographic image shooting devices 1 are loaded at the loading positions S1 and S2 or the loading positions S2 and S3. Nevertheless, the console C can determine at least which of the radiographic image shooting devices 1 (or the images p) is on the upper side (that is, “upper front side” or “upper lateral side”) and which of the radiographic image shooting devices 1 (or the images p) is on the lower side (that is, “lower front side” or “lower lateral side”).

Even when the number N of the images p acquired from the radiographic image shooting devices 1 is three or more, the console C can subject the images p to image analysis to determine accurately the vertical order of the radiographic image shooting devices 1 (or the images p).

Accordingly, even in the configuration of the modification example 3, the console C can determine the number N of the images p for use in the production of the long image plong based on the number of the images p transferred and acquired from the radiographic image shooting devices 1, and can determine accurately the vertical order (that is, the loading positions) of the radiographic image shooting devices 1 loaded in the holder 51a of the shooting stand 51A.

Needless to say, the present invention is not limited to the foregoing embodiment, modification examples, and the like, but can be modified as appropriate without deviating from the gist of the present invention.

Although the present invention has been described and illustrated in detail, it is clearly understood that the same is by way of illustrated and example only and is not to be taken by way of limitation, the scope of the present invention being interpreted by terms of the appended claims.

What is claimed is:

1. A radiographic image shooting system comprising:
a shooting stand configured to include a holder capable of being loaded with a plurality of radiographic image shooting devices;
an irradiation device configured to be capable of emitting radiation to the plurality of radiographic image shooting devices loaded in the holder; and
a console configured to control shooting based on selected shooting order information and composite images acquired from the plurality of radiographic image shooting devices to produce a long image, wherein
when the selected shooting order information is shooting order information for long shooting, the console creates a first shooting condition key for the long shooting,
the console determines the number of the images for use in the production of the long image, and creates newly a number of second shooting condition keys corresponding to the determined number of the images in association with loading positions of the radiographic image shooting devices having shot the images in the holder, and
the console links the images acquired from the radiographic image shooting devices with the second shooting condition keys corresponding to the loading positions of the radiographic image shooting devices, and links the long image produced by compositing the images with the first shooting condition key.

2. The radiographic image shooting system according to claim 1, comprising a detection unit configured to detect the presence or absence of loading of the radiographic image shooting devices in the holder and the loading positions, wherein
the console determines the number of the radiographic image shooting devices loaded in the holder at the time of radiation emission from the irradiation device based on the results of detection by the detection unit, and determines the number of the images for use in the production of the long image based on the determined number of the radiographic image shooting devices.

3. The radiographic image shooting system according to claim 1, comprising a detection unit configured to detect the presence or absence of loading of the radiographic image shooting devices in the holder, the loading positions, and whether the radiographic image shooting devices are to be used for shooting, wherein
the console determines the number of the radiographic image shooting devices capable of shooting out of the radiographic image shooting devices loaded in the holder at the time of radiation emission from the irradiation device based on the results of detection by the detection unit, and determines the number of the images for use in the production of the long image based on the determined number of the radiographic image shooting devices.

4. The radiographic image shooting system according to claim 1, wherein
the radiographic image shooting devices detect the loading positions in the holder, and
at the time of acquisition of the images from the plurality of radiographic image shooting devices, the console acquires information on the loading positions detected by the radiographic image shooting devices together with the images, and
the console determines the number of the images for use in the production of the long image based on the number of the images acquired from the radiographic image shooting devices, and determines the loading positions based on the information on the loading positions acquired together with the images.

5. The radiographic image shooting system according to claim 1, wherein
the console determines the number of the images for use in the production of the long image based on the number of the images acquired from the radiographic image shooting devices, and subjects the images to image analysis to determine the loading positions of the radiographic image shooting devices having shot the images in the holder.

6. The radiographic image shooting system according to claim 1, wherein the console establishes an association between the long image linked with the first shooting condition key and the images linked with the second shooting condition keys.

7. The radiographic image shooting system according to claim 1, wherein the console notifies that shooting is enabled only when all the plurality of radiographic image shooting devices loaded in the holder of the shooting stand becomes capable of shooting.

8. The radiographic image shooting system according to claim 1, wherein, when any of the plurality of radiographic image shooting devices loaded in the holder of the shooting stand is incapable of shooting, the console inhibits the emission of radiation from the irradiation device.

9. The radiographic image shooting system according to claim 1, wherein the console displays on a display unit only the first shooting condition key but does not display the second shooting condition keys.

10. The radiographic image shooting system according to claim 1, wherein the console displays on the display unit parameters for the loading positions of the radiographic image shooting devices loaded in the holder and/or shooting.

11. The radiographic image shooting system according to claim 1, wherein dongles storing different identification information are arranged in the holder of the shooting stand at the loading positions where the radiographic image shooting devices are capable of being loaded, and when the dongles are connected to the radiographic image shooting devices at the time of loading of the radiographic image shooting devices into the holder, the radiographic image shooting devices read the identification information stored in the dongles and send the same to the detection unit.
